(19) United States
(12) Patent Application Publication
(10) Pub. No.: US 2009/0186090 A1
(43) Pub. Date: Jul. 23, 2009

(54) Oral Care Composition to Reduce or Eliminate Dental Sensitivity

(75) Inventors: Lynette Zaidel, Cranford, NJ (US); Suman K. Chopra, Monroe, NJ (US); Michael Prencipe, Princeton Junction, NJ (US); Qin Wang, Monmouth Junction, NJ (US); Constantina Christopoulou, Somerset, NJ (US)

Correspondence Address: COLGATE-PALMOLIVE COMPANY, 909 RIVER ROAD, PISCATAWAY, NJ, 08855, US

(73) Assignee: COLGATE-PALMOLIVE, New York, NY (US)

(21) Appl. No.: 12/356,837

(22) Filed: Jan. 21, 2009

Related U.S. Application Data
(63) Continuation-in-part of application No. 12/339,598, filed on Dec. 19, 2008.
Application No. 12/339,598 is a continuation-in-part of application No. 12/103,919, filed on Apr. 16, 2008.
Application No. 12/103,919 is a continuation-in-part of application No. 11/742,039, filed on Apr. 30, 2007.

(51) Int. Cl. A61K 9/14; A61P 1/02

(52) U.S. Cl. 424/489

(57) ABSTRACT

The invention includes an oral care composition that reduces and/or eliminates the perception of tooth sensitivity. The composition includes an adherent material and includes, in part, particles having a particle size of 2-5 microns. Also included within the scope of the invention are methods comprising the use of such compositions, such as methods of reducing dental sensitivity.

### CROSS-REFERENCE TO RELATED APPLICATIONS

This application is a continuation-in-part of U.S. patent application Ser. No. 12/338,598, filed Dec. 18, 2008, which is a continuation-in-part of U.S. patent application Ser. No. 12/103,919, filed Apr. 16, 2008, which is a continuation-in-part of U.S. patent application Ser. No. 11/742,039, filed Apr. 30, 2007, each of which application is hereby incorporated by reference in its entirety.

### BACKGROUND OF THE INVENTION

Dentin is a portion of the tooth internal to the enamel and cementum that has a radially striated appearance owing to a large number of fine canals or tubules known as the dentinal tubules. Tubules run from the pulp cavity to the periphery of the dentin and are generally about two microns in diameter at their base and somewhat narrower at their periphery. Tubules are not usually exposed to the environment in the oral cavity, as they are usually covered by enamel or cementum. The cementum in turn is often covered by the gums.

It is commonly understood that partially or fully exposed tubules can lead to tooth sensitivity, an irritating and painful condition. In this theory, recession of the gum line exposes cementum to erosion. The eroded cementum in turn exposes the hollow dentinal tubules. The exposed tubules cause nerves within the tooth to be affected excessively by external oral stimuli because material and energy transfer between the exterior and interior of the tooth is accelerated through the tubules. Common environmental stimuli, such as heat, cold, chemicals and physical and mechanical pressure or stimuli, such as brushing, are able to irritate the nerve through the open dentin tubules and thereby create pain. The pain of sensitive teeth appears to result from these stimuli, which apparently cause fluid movements in the dentinal tubules that activate pulpal nerve endings.

Conventionally, two approaches have been taken to treat or ameliorate tooth sensitivity. Under one approach, the chemical environment proximal to the nerve is altered by application of various agents, such that the nerve is not stimulated, or not stimulated as greatly. Known agents useful in this chemical approach, including potassium salts (such as potassium nitrate, potassium bicarbonate, potassium chloride) and strontium, zinc salts, and chloride salts.

The second approach involves the mechanical shield of the nerve by, e.g., blocking of the dentinal tubules wholly or partially with “tubule blocking agents.” Agents that have been disclosed in the prior art include, e.g., cationic alumina, clays, water-soluble or water-swellable polyelectrolytes, oxalates, amorphous calcium phosphate, hydroxyapatite, maleic acid copolymers and polyethylene particles.

However, both the chemical and the mechanical approaches, because they require the incorporation of one or more additional materials to the dentifrice, may result in formulation difficulties, either technical or related to increased costs. For this reason there is a need in the art for a dentifrice that, upon use, prevents or reduces tooth sensitivity yet is not associated with significant processing or formulation disadvantages.

### BRIEF SUMMARY OF THE INVENTION

The invention includes an oral care composition comprising an adherent material and silica particles, wherein the oral care composition provides a fluid floss rate of no greater than about 45% of the fluid floss rate of etched dentin. In an aspect, a composition comprises silica particles having a particle size distribution (PSD) of 3 μm to 5 μm. In another aspect, a composition comprises silica particles having a median particle size of 3 μm to 5 μm. In yet another aspect, the composition comprises silica particles having an average particle size of 3 μm to 5 μm. In an aspect of the invention, a composition comprises silica particles having a particle size distribution (PSD) of 2 μm to 5 μm. In another aspect, a composition comprises silica particles having a median particle size of 2 μm to 5 μm. In yet another aspect, the composition comprises silica particles having an average particle size of 2 μm to 5 μm. In another aspect, a composition comprises silica particles having an average particle size of 2.7 μm to 4.0 μm. In another aspect, a composition comprises a population of silica particles having a particle size selected from the group consisting of 2 μm, 2.5 μm, 3 μm, 3.5 μm, 4 μm, 4.5 μM, and 5 μm, wherein said population of silica particles comprise at least 20% of the total silica particles in said oral care composition. In another aspect, a composition comprises silica particles having a median particle size of 3 μm to 5 μm, a d10 of 1.5 μm to 3 μm, and a d90 of 6 μm to 11 μm. In yet another aspect, a composition comprises silica particles having a median particle size of 2 μm to 4 μm, a d10 of 0.5 μm to 2 μm, and a d90 of 5 μm to 10 μm.

In an aspect of the invention, a composition comprises silica particles, wherein the composition has a cumulative particle size volume fraction≦3.95 μm (AUC 3.95) of at least 20%, and wherein the oral care composition provides a fluid flow rate of no greater than about 45% of the fluid flow rate of etched dentin. In another aspect, a composition comprises silica particles, wherein the silica particles comprise a population of starting material silica particles having a cumulative particle size volume fraction (AUC 3.95) of at least 40%, wherein the oral care composition provides a fluid flow rate of no greater than about 45% of the fluid flow rate of etched dentin.

In an aspect, the silica particles of a composition have a porosity of less than 0.45 cc/g in pores of 600 Angstroms or smaller.

In an aspect, the adherent material in a composition is a polymer having a number average molecular weight between 100,000 and 2,500,000, inclusive. In an aspect, the adherent material is selected from polymers of polyvinyl phosphonic acid, poly (l-phosphonopropene) sulfonic acid, poly(beta styrene phosphonic acid), alpha styrene phosphonic acid, synthetic anionic polymeric polycarboxylate, maleic anhydride, maleic acid, and methyl vinyl ether. In another aspect, the adherent molecule is a polymer of methyl vinyl ether and maleic anhydride.

In an aspect of the invention, a composition is formulated into a form selected from a rinse, a paste, a gel, a gum, a dissolvable lozenge, and a film. In another aspect, the composition is formulated into a form selected from a dissolvable film.

In an aspect of the invention, a composition comprises a non-silica desensitizing agent. In an aspect, the desensitizing agent is selected from the group consisting of a nitrate salt, an arginine ester, a bicarbonate salt, potassium nitrate, potassium chloride, an arginine-bicarbonate-phytate complex, potassium citrate, and arginine.

In an aspect, a composition further comprises an antibacterial agent. In an aspect, a composition further comprises 2,4,4′-trichloro-2′-hydroxydiphenyl ether.

In an aspect, a composition further comprises an agent selected from a chemical whitening agent, an opaque whitening agent and an anticalculus agent. In an aspect, a composition further comprises a surfactant system that comprises sodium lauryl sulfate and tauranol. In an aspect, a surfactant system consists essentially of sodium laurel sulfate and tauranol in a ratio of 1:5 to 1:3.

In an aspect, a composition further comprises an agent selected from a stannous ion agent; a fluoride compound; sodium fluoride; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethyl pyridinium chloride (TDEPC); octenidine; delmopinol; octapinol; nisin; zinc ion agent; copper ion agent; essential oils; furanones; bacteriocins, ethyllauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, and propolis.

In an aspect, the invention provides compositions and methods for reducing dental sensitivity. In an aspect, a method of reducing dental sensitivity comprises applying to the surface of a mammalian tooth an oral care composition of provided for herein. In another aspect, a method of reducing dental sensitivity comprises applying to the surface of a mammalian tooth an oral care composition of claim 1, wherein the adherent material is selected from polymers of polyvinyl phosphonic acid, poly (l-phosphonopropene) sulfonic acid, poly(beta styrene phosphonic acid), alpha styrene phosphonic acid, synthetic anionic polymeric polycarboxylate, maleic anhydride, maleic acid, and methyl vinyl ether. In an aspect, a method of reducing dental sensitivity comprises applying to the surface of a mammalian tooth an oral care composition as provided for herein, wherein the particles have a porosity of less than 0.45 cc/g in pores of 600 Angstroms or smaller.

In an aspect, a method is provided for protecting dentin from acid-mediated degradation, comprising applying to the surface of a mammalian tooth an oral care composition as provided for herein.

In another aspect, a method is provided for maintaining or increasing the systemic health of a mammal comprising applying a composition to an oral surface of a mammal at least once a day for a duration of time, wherein the composition comprises an oral care composition as provided for herein, wherein the silica particles are present in the composition in an amount of 5% by weight or greater, and an agent selected from triclosan; triclosan monophosphate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; delmopinol; octapinol; nisin; zinc ion agent; copper ion agent; essential oils; furanones; bacteriocins, ethyllauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, and propolis.

Also included is a method of occluding a dentin tubule within the surface of a mammalian tooth, comprising applying to the tooth surface a composition comprising an adherent material and a silica particle having a median particle size of no greater than a dentin tubule. In an aspect, a method of occluding a dentin tubule within the surface of a mammalian tooth comprises applying to the tooth surface a composition as provided for herein. In an aspect, the method of application is a method other than brushing the tooth surface. In another aspect, a method of desensitizing a tooth in less than one day is provided, the method comprising applying to the tooth surface a composition as provided for herein.

The invention includes a method of increasing the potassium flux of a tooth, the method comprising applying to the tooth surface a composition as provided for herein. Also included is a method of increasing the potassium flux of a conventional potassium-containing desensitizing dentifrice, the method comprising applying to the tooth surface a composition as provided for herein. In an aspect, a method of increasing the potassium flux of a conventional potassium-containing desensitizing dentifrice comprises applying to the tooth surface the composition as provided for herein, wherein the composition is applied either prior to application of the conventional dentifrice to the tooth, concomitant with application of the conventional dentifrice to the tooth, concomitant with application of the conventional dentifrice to the tooth in a mixture with the conventional dentifrice, or bib way of any combination thereof.

### BRIEF DESCRIPTION OF THE DRAWINGS

FIG. 1 depicts a comparison of the occlusion incidence resulting from treating with an oral care composition of the invention versus two different conventional desensitizing dentifrices in an acid-treated mammalian tooth dentin substrate. Standard silica-containing desensitizing dentifrices are shown in columns 1 and 3, and a silica-containing dentifrice of the invention is shown in column 2.

FIG. 2 depicts the reduction in hydraulic conductance (% occlusion) of dentin segments treated with an oral care composition of the invention versus those treated with a conventional dentifrice.

FIG. 3 is a schematic depicting the potassium flux experiment of Example 3.

FIG. 4 depicts the difference in measured potassium flux with an oral care composition of the invention versus a conventional dentifrice.

FIG. 5 depicts potassium flux under simulated pulpal pressure of 20 cm H₂0.

FIG. 6 depicts the change in potassium flux upon change of pulpal pressure from 0 cm H₂0 to 20 cm H₂0.

### DETAILED DESCRIPTION OF THE INVENTION

The invention described herein includes an oral care composition that contains at least (a) an adherent material and (b) a silica particle. The silica particle may have an average particle size of no greater than a dentin tubule, or alternatively it may have a median particle size of 8 microns or less. The particles may be present in an amount of 5% by weight or greater. The compositions may contain additional therapeutic and non-therapeutic components, and may also be utilized in the practice of various methods, all of which are included within the scope of the invention. The composition and methods within the scope of the invention may be useful in, for example, reducing or eliminating tooth sensitivity of a mammal, improving/maintaining systemic health, and/or occluding dentin tubules.

Particle size distribution is measured using a Malvern Particle Size Analyzer, Model Mastersizer 2000 (or comparable model) (Malvern Instruments, Inc., Southborough, Mass.), wherein a helium-neon gas laser beam is projected through a transparent cell which contains silica, such as, for example, silica hydrogel particles suspended in an aqueous solution. Light rays which strike the particles are scattered through angles which are inversely proportional to the particle size. The photodetector arrant measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the silica hydrogel, for example. It will be understood that other methods of measuring particle size are known in the art, and based on the disclosure set forth herein, the skilled artisan will understand how to calculate median particle size, mean particle size, and/or particle size distribution of silica particles of the present invention.

Silicas and silica compositions. In an aspect, suitable silica particles for oral compositions of the invention include silica particles with, for example, a particle size distribution of 3 to 4 microns, or alternatively, a particle size distribution of 5 to 7 microns, alternatively, a particle size distribution of 3 to 5 microns, alternatively, a particle size distribution of 2 to 5 microns, or alternatively, a particle size distribution of 2 to 4 microns.

The oral compositions within the scope of the invention also include particles that have a median particle size that is no greater than the average diameter of a mammalian dentin tubule, such that one or more particles is/are capable of becoming lodged within the tubule, thereby effecting a reduction or elimination of perceived tooth sensitivity.

In an aspect, suitable silica particles may have, for example, a median particle size of 8 microns or less, alternatively, a median particle size of 3 to 4 microns, alternatively, a median particle size of 5 to 7 microns, alternatively, a median particle size of 3 to 5 microns, alternatively, a median particle size of 2 to 5 microns, or alternatively, a median particle size of 2 to 4 microns.

In an embodiment, a silica particle has a particle size of 2.0 microns. In another embodiment, a silica particle has a particle size of 2.5 microns. In another embodiment, a silica particle has a particle size of 3.0 microns. In another embodiment, a silica particle has a particle size of 3.5 nm microns. In another embodiment, a silica particle has a particle size of 4.0 microns. In another embodiment, a silica particle has a particle size of 4.5 microns. In another embodiment, a silica particle has a particle size of 5.0 microns. In an aspect of the invention, the silica particle size is a median particle size. In another aspect, the silica particle size is an average (mean) particle size. In an embodiment, the silica particle comprises at least 5%, at least 10%, at least 15%, at least 20%, at least 25%, at least 30%, at least 35%, or at least 40% of the total silica particles in a silica particle-containing composition.

In an aspect of the invention, a silica has a particle size characterized by the parameters of a median particle size of about 2 μm to about 4 μm, a d₁₀ of about 0.5 μm to about 2 μm, and a d₉₀ of about 5 μm to about 10 μm. As used herein, d₁₀ refers to particles having a diameter that is 10% of the threshold of the sampled population (i.e., 10% of the population is equal to or smaller than the d₁₀ value), and d₉₀ refers to particles having a diameter that is 90% of the threshold of the sampled population (i.e., 90% of the population is equal to or smaller than the d90 value). In another aspect, a silica has a particle size characterized by a median particle size of about 3 μm to about 5 μm, a d₁₀ of about 1.5 μm to about 3 μm, and a d₉₀ of about 6 μm to about 11 μm.

In another aspect of the invention, at least a portion of the silica in a silica-containing dentifrice has a d₅₀ of 3.95 μm (i.e., 50% of the population of silica particles is equal to or smaller than the d₅₀ value). Sorbosil AC43 silica has a d₅₀ of 3.95 μm. By way of a non-limiting example, the d₅₀ is measured using particle size measuring techniques as set forth elsewhere herein (e.g., MALVERN MASTERSIZER). In an embodiment, a silica-containing dentifrice has a population of particles at and below 3.95 μm as determined by the area under the curve (AUC) obtained in a particle size measurement. As used herein, the term “AUC 3.95” refers to the cumulative volume fraction of particles≦3.95 μm. By way of a non-limiting example, a composition having 20% of its particles≦3.95 μm is said to have a cumulative particle size volume fraction (AUC 3.95) of 20%.

In an embodiment, a silica-containing dentifrice of the invention has an AUC 3.95 value of least 18%. In another embodiment, a silica-containing dentifrice of the invention comprises has an AUC 3.95 value of least 26%. In another embodiment, a silica-containing dentifrice of the invention comprises has an AUC 3.95 value of least 22%. In another embodiment, a silica-containing dentifrice of the invention has an AUC 3.95 value of least 24%. In another embodiment, a silica-containing dentifrice of the invention has an AUC 3.95 value of least 26%. In another embodiment, a silica-containing dentifrice of the invention has an AUC 3.95 value of least 30%.

In another embodiment, a silica-containing dentifrice of the invention comprises a silica starting material that has an AUC 3.95 value of at least 40%. In another embodiment, a silica-containing dentifrice of the invention comprises a silica starting material that has an AUC 3.95 value of at least 42%. In another embodiment, a silica-containing dentifrice of the invention comprises a silica starting material that has an AUC 3.95 value of at least 45%. In another embodiment, a silica-containing dentifrice of the invention comprises a silica starting material that has an AUC 3.95 value of at least 50%. In an aspect of the invention, a silica starting material is a small particle silica.

In an aspect of the invention, the silica particles have a porosity of less than about 0.45 cc/g in pores of about 600 Angstroms or smaller.

In an embodiment, the silica is an INEOS (now PQ Corp.) Sorbosil AC43 silica. In an embodiment, AC43 silica has properties including, but not limited to, an average particle size of 2.7-4.0 microns (as determined by MALVERN MASTERSIZER), a sieve residue of +45 μm, a moisture loss at 105° C. of 8.0% max, an ignition loss at 1000° C. of 14.0% max, and a pH of 5.5-7.5 in aqueous suspension.

In an embodiment, the silica particles may be initially present in the composition having the desired particle size, or may be initially present in the composition at a larger size, so long as the structure of the particles is such that it fractures or breaks into the desired particle size upon application of mechanical force by, e.g., a toothbrush, when brushing.

The silica particle may be prepared by any means known or to be developed in the art, and may be surface modified, if desired, to increase the capacity of the particle to adhere to a tooth surface. Examples may be found in, e.g., U.S. patent application Ser. No. 11/271,306, the contents of which are incorporated herein by reference. The silica particle is present in the composition in an amount of 5% or greater by weight of the total composition. Alternatively, the silica particle may be present in an amount of 5%, 6%, 7%, 8%, 9%, 10%, 15%, 20% or 25% by a weight.

Any abrasive particulates may be used and may be selected from sodium bicarbonate, calcium phosphate (e.g., dicalcium phosphate dihydrate), calcium sulfate, precipitated calcium carbonate, silica (e.g., hydrated silica), iron oxide, aluminium oxide, perlite, plastic particles, e.g., polyethylene, and combinations thereof. In particular, the abrasive may be selected from a calcium phosphate (e.g., dicalcium phosphate dihydrate), calcium sulfate, precipitated calcium carbonate, silica (e.g., hydrated silica), calcium pyrophosphate and combinations. Any type of silica may be used, such as precipitated silicas or silica gels. In an embodiment, commercially available silicas are used, such as INEOS AC43, available from Ineos Silicas, Warrington, United Kingdom, as described elsewhere herein. In an embodiment, a silica has a median particle size from 3 μm to 5 μm, as described in detail elsewhere herein. In another embodiment, a silica and/or silica-containing oral composition provides a fluid flow rate of no greater than about 45% of the fluid flow rate of etched dentin, as described in detail elsewhere herein.

Various abrasives may be used in accordance with the present invention. One class of abrasives comprises silica particles as set forth in detail herein. Another class of abrasives are powdered silicas, particularly, silica xerogels as defined in U.S. Pat. No. 3,538,230. Additionally, as set forth in U.S. Pat. No. 4,358,437, powdered forms of calcium carbonate in an abrasive form is another class of abrasives.

Polymers and adherent materials. The oral compositions of the invention also include an adherent material. The adherent material may be any known or to be developed in the art that attaches to the surface of a mammalian tooth and/or to the heterogenous biofilm which also may be present on a tooth's surface. Attachment may occur by any means, such as ionic interaction, van der Waals forces, hydrophobic-hydrophilic interactions, etc. The adherent material may be, for example, chitosan, chitin, a gum or a marine colloid. Other contemplated adherent materials include any homopolymers or copolymers (hereinafter referred to collectively as a “polymers”) that adhere to the surface of a tooth. Such polymers may include poly (ethylene oxide) polymers (such as POLYOX from Dow Chemical), linear PVP and cross-linked PVP, PEG/PPG copolymers (such as BASF Pluracare L1220), ester gum, shellac, pressure sensitive silicone adhesives (such as BioPSA from Dow-Corning), methacrylates, or mixtures thereof. In an embodiment, a copolymer comprises poly(methylvinylether/maleic anhydride). In another embodiment, a copolymer comprises poly(methylvinylether/maleic acid). In another embodiment, a copolymer comprises poly(methylvinylether/maleic acid) half esters. In another embodiment, a copolymer comprises poly(methylvinylether/maleic acid) mixed salts.

Polymers of any molecular weight may be used, including, for example molecular weights of 50,000 to 500,000, 500,000 to 2,500,000 or 2,500,000 to 10,000,000 (calculated by either number average or weight average). In an embodiment, a polymer has a molecular weight of 130,000. In an embodiment, a polymer has a molecular weight of 200,000. In an embodiment, a polymer has a molecular weight of 690,000. In an embodiment, a polymer has a molecular weight of 1,000,000. In an embodiment, a polymer has a molecular weight of 1,250,000. In an embodiment, a polymer has a molecular weight of 1,980,000. In another embodiment, a polymer has a molecular weight of 2,500,000. In yet another embodiment, a polymer has a molecular weight of 5,000,000.

In an embodiment, a copolymer of methyl vinyl ether and maleic anhydride may be used at a monomer ratio of from 1:4 to 4:1. Other polymers that may be used as adherent materials include those recited in United States Patent Application Publication No. 2006/0024246, the contents of which is incorporated herein by reference.

Commercially-available polymers may be used in the present invention. It is understood that over time, the exact size, weight and/or composition of a commercially-available polymer may change. Based on the disclosure set forth herein, the skilled artisan will understand how to determine whether such polymers are useful in the invention.

Dentin Conductance Evaluation: Dentin that is treated with the combination of the invention produce a fluid flow rate of no greater than 45%, 25%, 20%, 15% or 10% of the flow rate value of the etched dentin, as determined by the Dentin Conductance Procedure.

Dentin Conductance Procedure: The reduction in sensitivity of a tooth is demonstrated herein by a reduction in the measured fluid flow rate, a measure of conductance of dentin.

Extracted human molars are cut at the crown and roots using a diamond saw. The pulp is removed and the resulting dentin segment is stably mounted, such as onto an acrylic block. Tubing is connected from a hole in the acrylic block mounting just below %, the pulp chamber. The dentin segment is connected to an apparatus that measures the rate of fluid flour (hydraulic conductance). See, Zhang et al., “The effects of pain free desensitizer on dentine permeability and tubule occlusion over time, in vitro”, Journal of Clinical Periodontol, 25(11 Pt 1): 884-91 (November 1998), the contents of which are incorporated herein by reference.

The top surface of the dentin is etched with citric acid. The fluid flow rate across the etched dentin is measured under 70 cm water pressure. The dentin surface is then treated with a slurry of the oral composition of the invention diluted with 3 parts deionized water and the fluid flow rate is measured again. See Pashley et al., “Effects of desensitizing dentifrices in vitro,” J. Periodontol., 55 (9): 522-525 (September 1984).

Desensitizing Silica Compositions

In an aspect, silica-containing compositions of the invention can desensitize a tooth. In another aspect, silica-containing compositions of the invention provide tooth desensitization that is superior to conventional desensitizing dentifrices. By way of a non-limiting example, a silica-containing dentifrice of the invention provides tooth desensitization by providing a greater desensitization than a conventional dentifrice or a conventional desensitizing dentifrice, by providing desensitization more rapidly than a conventional dentifrice or a conventional desensitizing dentifrice, or by a combination of greater desensitization and more rapid desensitization, among other effects. In an embodiment, a silica-containing composition of the invention provides desensitization and/or superior desensitization in the absence of any other desensitizing agent. In another embodiment, a silica-containing composition of the invention provides desensitization and/or superior desensitization, and may contain one or more additional desensitizing agents, as described elsewhere herein.

The invention also encompasses methods of use and/or application of a silica-containing desensitizing composition. In an embodiment, a silica-containing composition may be applied to the tooth via conventional brushing techniques (e.g., use of a toothbrush). In another embodiment, a silica-containing composition may be applied to the tooth via a method other than conventional brushing techniques. Other methods of application include manual application (e.g., applying a composition to a tooth using one or more fingers, rubbing onto the tooth surface, rubbing in a circular motion, etc. . . . ), or application using any known dental appliance or applicator. It will be understood, based on the disclosure set forth herein, that any method of smearing a composition onto a tooth, optionally using varying degrees of physical pressure, is encompassed by the invention.

Desensitization of a tooth according to the invention may be measured by any technique set forth herein, or any technique known to the skilled artisan. In an embodiment, the extent of desensitization of a tooth according to a composition of the invention may be ascertained by measuring the potassium flux, as described in detail elsewhere herein.

Additionally, the invention provides compositions and methods for augmenting, enhancing and/or supplementing the desensitization obtained using potassium-based desensitizing dentifrices. In an aspect, a composition of the invention is used to occlude a dentin tubule to inhibit outward fluid flow while at the same time allowing inward flux of potassium ions into the tubule. In another aspect, the invention provides compositions and methods of desensitizing a tooth, wherein the degree of desensitization is ascertained by measuring potassium flux as 20 cm pulpal pressure, as described in detail elsewhere herein. In an embodiment, a composition of the invention has a potassium flux value at 20 cm pulpal pressure that is greater than 20% of the potassium flux value obtained for the composition at zero pulpal pressure.

Surprisingly, it was found that the combination of the small particle silica/polymer occlusion composition with a potassium desensitizing agent enhanced the delivery of potassium inward through the dentin tubules. In an embodiment, the invention provides compositions and methods for increasing the potassium flux value measured upon use of a conventional potassium-containing dentifrice applied to a tooth. Such a potassium flux change can be measured as described in detail elsewhere herein. The invention encompasses any increase of the potassium flux obtained when using a conventional potassium-containing dentifrice, as mediated by co-application of a conventional potassium-containing dentifrice and a silica-containing composition of the invention, by application of a conventional potassium-containing dentifrice subsequent to application of a silica-containing composition of the invention, or by application of a mixture comprising a conventional potassium-containing dentifrice and a silica-containing composition of the invention.

The invention includes a method of increasing the potassium flux in one or more dentin tubules of a tooth, the method comprising applying to the tooth surface a composition as provided for herein. Application of the composition to the tooth surface results in the introduction of the composition into one or more dentin tubules. The composition is applied to the teeth by any method set forth herein or known in the art. The potassium flux, the rate of potassium flux, and the change in rate of potassium flux can be ascertained as set forth in detail elsewhere herein.

Also included is a method of increasing the potassium flux of a conventional potassium-containing desensitizing dentifrice, the method comprising applying to the tooth surface a composition as provided for herein. In an aspect, a method of increasing the potassium flux of a conventional potassium-containing desensitizing dentifrice comprises applying to the tooth surface the composition as provided for herein, wherein the composition is applied either prior to application of the conventional dentifrice to the tooth, concomitant with application of the conventional dentifrice to the tooth, concomitant with application of the conventional dentifrice to the tooth in a mixture with the conventional dentifrice, or by way of any combination thereof.

Oral Care Compositions: The oral care composition may include any other therapeutic, cosmetic, and/or aesthetic materials as may be desired. Examples include non-silica desensitizing agents (E.g., a nitrate salt, an arginine ester, a bicarbonate salt, potassium nitrate, an arginine-bicarbonate-phytate complex, potassium citrate, and arginine, among others), a chemical whitening agent (such as a peroxide releasing compound), an opaque whitening agent (such as hydroxyapetite) and an anticalculus agent. Other options for inclusion in the oral care composition of the invention include triclosan; stannous ion agents; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; delmopinol; octapinol; nisin; zinc ion agents; copper ion agents; essential oils; furanones; bacteriocins, ethyl lauroyl arginate, extracts of magnolia, a metal ion source, arginine bicarbonate, honokiol, magonol, ursolic acid, ursic acid, morin, extract of sea buckthorn, an enzyme, a Camellia extract, a flavonoid, a flavan, halogenated diphenyl ether, creatine, and propolis.

The oral care compositions described herein may be formulated into any delivery form that permits contact of the adherent material and the particles, to the tooth surface. For example, the compositions may be formulated into a mouth rinse, a paste, a gel, a lozenge (dissolvable or chewable), a spray, a gum, and a film (wholly or partially dissolvable, or indissoluble). The composition may contain any conventional excipients or carriers, although these will vary depending on the dosage form or means of dosage selected. Excipents or carriers can include, for example, humectants, colorants, flavorants, glycerin, sorbitol, xylitol, and/or propylene glycol, water or other solvents, gum bases, thickening agents, surfactants, carrageenan (rich moss), xanthan gum and sodium carboxymethyl cellulose, starch, polyvinyl pyrrolidone, hydroxyethyl propyl cellulose, hydroxybutyl methyl cellulose, hydroxypropyl methyl cellulose, and hydroxylethyl cellulose and amorphous silicas.

Surfactants may be included, if desired. Examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of the last mentioned amides include N-lauryl sarcosine, and the sodium, potassium and ethanolamine salts of N-lauryl, N-myristoyl, or N-palmitoyl sarcosine. Others include, for example, nonanionic polyoxyethylene surfactants, such as Polyoxamer 407, Steareth 30, Polysorbate 20, and castor oil; and amphoteric surfactants, such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from 12 to 20 carbon atoms), which condensation products (ethoxamers) contain hydrophilic polyoxyethylene moieties, such as condensation products of poly (ethylene oxide) with fatty acids, fatty, alcohols, fatty amides and other fatty moieties, and with propylene oxide and polypropylene oxides.

In an embodiment, the oral composition includes a surfactant system that is sodium laurel sulfate (SLS) and tauranol. If desired, the SLS and tauranol may be present in a ratio of 1:5 to 1:3.

The oral care composition of the invention may be prepared by any means known in the art. For example, preparation methods for dentifrices are well known, for example, as described in U.S. Pat. Nos. 3,966,863; 3,980,767; 4,328,205; and 4,358,437, the contents of which are incorporated herein by reference. In general, any humectant (e.g., glycerin, sorbitol, propylene glycol, and/or polyethylene glycol) is dispersed in water in a conventional mixer under agitation. Into that dispersion are added the thickeners, such as carboxylmethyl cellulose (CMC), carrageenan, or xanthan gum; any anionic polycarboxylate; any salts, such as sodium fluoride anticaries agents; and any sweeteners.

The resultant mixture is agitated until a homogeneous gel phase is formed. Into the gel phase are added any pigments utilized, such as TiO₂, and additionally any acid or base required to adjust the pH of the composition. These ingredients are mixed until a homogeneous phase is obtained.

The mixture is then transferred to a high speed/vacuum mixer, wherein the surfactant ingredients are added to the mixture. The silicas utilized are added subsequently. Any water insoluble agents, such as triclosan, are solubilized in the flavor oils to be included in the dentifrice, and that solution is added along with the surfactants to the mixture, which is then mixed at high speed in the range from 5 to 30 minutes, under a vacuum of 20 to 50 mm of Hg. The resultant product is a homogeneous, semi-solid, extrudable paste or gel product.

Methods of use: The invention also includes within its scope several related methods. For example, the invention includes within its scope methods of reducing and methods of occluding a dentin tubule of a mammalian tooth, methods of protecting dentin from acid-mediated degradation, and methods of reducing dental sensitivity.

Each of these methods includes the steps of applying any of the compositions described above to the tooth surface. Application may be carried out by any method, so long as the adherent material and the particles are placed in contact with the tooth surface. Application may be accomplished by brushing, flossing, prophylaxis, irrigating, wiping, rinsing (lavage of oral cavity), foam/gel and in-tray application, masticating, spraying, painting, etc., or applied by film or strip.

Dental sensitivity may be reduced according to a method of the invention by applying a composition of the invention to a tooth surface. A composition may be applied using a traditional method, as described in detail elsewhere herein, or by any appliance or applicator, whether or not typically associated with dental use. In an embodiment, one or more human fingers is used to apply a dental sensitivity-reducing composition to one or more teeth. A finger can be used to smear the composition on the surface of a tooth, or to otherwise apply the composition to the surface of a tooth.

Alternatively, the invention includes methods to increase or maintain the systemic health of a mammal by applying a composite to an oral surface (both hard and soft tissues of the oral cavity). The composition for use in this method may be any described above, provided that it contains at least one of triclosan; triclosan monophosphate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; delmopinol; octapinol; nisin; zinc ion agent; copper ion agent; essential oils; furanones; bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, and propolis. The application may be at least once a day, although up to five times per day may be preferred, and may be carried out over a duration of time, e.g., one week, up to one year, up to three years or for a lifetime.

### Example 1

Four compositions in paste-form were prepared using the materials and amounts set out in Table 1 and the process described below. Composition A and is a control composition that does not contain the specified silica particle.

TABLE 1
Components included in tested compositions.

| Ingredient | A | B | C | D |
|---|---|---|---|---|
| Water | QS | QS | QS | QS |
| Saccharin | 0.3 | 0.3 | 0.3 | 0.3 |
| NaF | 0.243 | 0.243 | 0.243 | 0.243 |
| Glycerin | 20 | 20 | 20 | 20 |
| Propylene Glycol | 0.5 | 0.5 | 0.5 | 0.5 |
| Carboxy methyl cellulose (CMC) | 1.1 | 1.1 | 1.1 | 1.1 |
| Iota Carrageenan | 0.4 | 0.4 | 0.4 | 0.4 |
| TiO2 | 0.5 | 0.5 | 0.5 | 0.5 |
| Sorbitol | 20.85 | 20.85 | 20.85 | 20.85 |
| PMV/MA Copolymer 13% soln | 15 | 15 | 15 | 15 |
| NaOH | 1.2 | 1.2 | 1.2 | 1.2 |
| Thickening silicas | 1.5 | 1.5 | 1.5 | 1.5 |
| Abrasive silicas | 20 | 17 | 15 | 11 |
| Ineos AC43 small particle silica | 0 | 3 | 5 | 9 |
| Flavor component | 1 | 1 | 1 | 1 |
| triclosan | 0.3 | 0.3 | 0.3 | 0.3 |
| Sodium laureth sulfate | 1.5 | 1.5 | 1.5 | 1.5 |
| Total | 100 | 100 | 100 | 100 |

Sodium saccharin and sodium fluoride was dissolved in water. Triclosan was dissolved in the flavor component.

Glycerin and propylene glycol were mixed together. Sodium CMC and iota carragenan was dispersed. Titanium dioxide was added to the mixture. This was followed by the addition of sorbitol. To this sodium saccharin and sodium fluoride in water was added and it was mixed for 15 minutes at 49° C. Then the PMV/MA copolymer and sodium hydroxide (50%) were added at 49° C. (5 minutes mixing). The whole mixture was dropped into a mixer and mixed. Subsequently, the abrasive silicas and the Ineos AC43 silica particles were added at high speed under full vacuum.

Premix flavor and triclosan and sodium sulphate powder were added. It was mixed for 10 minutes at medium speed under full vacuum. The vacuum was released and the whole batch was inspected for uniformity.

Fluid flow across dentin samples using each composition (A-D) was measured using the procedure described above.

TABLE 2
Measured fluid flow values for prepared compositions

| | % Flow vs. |
|---|---|
| Composition | etched baseline |
| A (0% AC43 silica) | 92 ± 2 |
| B (3% AC43 silica) | 77 ± 8 |
| C (5% AC43 silica) | 22 ± 4 |
| D (9% AC43 silica) | 5 ± 1 |

Dentin treated with compositions C-D (polymer and small particle silica) produced a fluid flow rate that was 5-22% of the fluid flow value of etched dentin which was significantly lower than that of composition A with polymer alone. Values for tropical commercial dentifrices without the small particle silica/polymer would be 50-100% of the value of etched dentin (ref: Pashley D H et al, Effect of desensitizing dentrifices. J. Periodontol, 1984:55:522-525). Thus, compositions C-D produced significant reductions in fluid flow rate.

This observed reduction in conductance is a measure of the reduction in dental sensitivity. While not wishing to be bound by any particular theory, at least a partial occlusion of dentin tubules by a silica-containing oral care composition of the invention contributes to this reduction in dental sensitivity.

Similarly, confocal microscopy images taken of etched dentin treated with Composition C showed significant occlusion/coating of the open dentin tubules when compared to etched dentin treated with Composition A. In addition, the occlusive coating produced by Composition C was resistant to acid dissolution by cola.

### Example 2

### Effect of Triclosan/Copolymer/Small Particle Silica/NaF Dentifrice on Acid Erosion of Dentin

The ability, of an oral composition comprising triclosan/copolymer/small particle silica/NaF was examined for the protection of dentin from acid attack. Human dentin blocks were cut from extracted molars and masked with nail polish leaving only the occlusal surface exposed. Dentin surface area was measured (cm²) and the blocks were etched (one minute, 6% citric acid) and placed in phosphate buffered saline (PBS) for 5 minutes with ultrasonication. Duplicate dentin blocks were divided into three groups and treated for one minute with either PBS, a conventional dentifrice, or the Test Dentifrice set forth herein, comprising triclosan/copolymer/small particle silica/NaF, wherein the silica particle size distribution was between 2 μm and 8 μm. Both the conventional dentifrice and the Test Dentifrice according to the present invention contained 1100 ppm NaF. The dentin blocks were rinsed and incubated in PBS for 30 minutes. The cycle was repeated for a total of 6 treatments, followed by a 3-minute acid challenge in 6% citric acid. The citric acid challenge solution was analyzed for soluble calcium concentration with atomic absorption spectrophotometry.

All 3 treatment groups of dentin blocks were statistically different (p<0.05, one-way ANOVA, Tukey's T-test) for loss in calcium/cm² with dentin treated with the triclosan/copolymer/small particle silica/NaF oral composition of the invention exhibiting the lowest amount of calcium loss (see Table 3).

TABLE 3
Calcium loss based on treatment

| | Calcium |
|---|---|
| Treatment | ppm/cm² (st dev)^{sig} |
| PBS control | 229.0 (3.4) |
| Conventional dentifrice | 215.3 (2.0) |
| Triclosan/copolymer/small particle | 192.9 (2.0) |
| silica/NaF oral composition | |

Dentin treated with the triclosan/copolymer/small particle silica/NaF oral composition of the present invention provided significantly better protection against acid attack compared to dentin treated with a conventional fluoride dentifrice.

### Example 3

### Clinical Study on Hypersensitivity Reduction Efficacy of a Triclosan/Copolymer/Small Particle Silica/NaF Dentifrice

The objective of this eight-week, double-blind, parallel-group clinical stud), was to investigate the efficacy of a dentifrice containing 0.3% triclosan, 2.0% polymethylvinylether/maleic anhydride (PVM/MA) copolymer, small particle silica and 0.243% sodium fluoride in a silica base for the reduction of dentinal hypersensitivity.

Following a baseline hypersensitivity examination, eighty-two qualifying adults were randomized into two treatment groups balanced for hypersensitivity scores to tactile and air-blast stimuli: (1) a dentifrice containing 0.3% triclosan, 2.0% PVM/NIA copolymer, small particle silica and 0.243% NaF in a silica base (Test Dentifrice); and (2) a conventional commercially-available non-desensitizing dentifrice containing 0.243% NaF in a silica base (Control Dentifrice). Subjects were instructed to brush their teeth at home for one minute, twice daily (mornings and evenings), using only their assigned dentifrice product and provided soft-bristled adult toothbrush. Hypersensitivity examinations were repeated after four and eight weeks of product use.

Eighty-two subjects completed all study visits. At both the four-week and the eight-week examinations, the Test Dentifrice group exhibited statistically significantly more favorable tactile hypersensitivity scores than did the Control Dentifrice group, with improvements of 31.6% and 52.1%, respectively. Additionally, at both the four-week and eight-week examinations, the Test Dentifrice group exhibited statistically significantly more favorable air blast hypersensitivity scores than did the Control Dentifrice group, with improvements of 17.8% and 23.6%, respectively.

The results of this clinical study support the conclusions that an oral composition of the invention, in the form of a dentifrice containing 0.3% triclosan, 2.0% copolymer, small particle silica and 0.243% NaF in a silica base provides (1) a significant reduction of hypersensitivity after four and eight weeks of product use, and provides (2) significant improvements in dentinal hypersensitivity as compared to the commercially-available non-desensitizing fluoride dentifrice after four and eight weeks of product use.

### Example 4

### The Hypersensitivity Reduction Efficacy of a Triclosan/Copolymer/Small Particle Silica/Sodium Fluoride Dentifrice: A Multi-Site Clinical Study

The objective of this double-blind, parallel-group clinical study, conducted at six independent investigational sites was to investigate the efficacy of a dentifrice containing 0.3% triclosan, 2.0% PVM/MA copolymer, small particle silica and 0.243% sodium fluoride for the reduction of dentinal hypersensitivity

Following a baseline hypersensitivity examination, 366 qualifying adults were randomized into two treatment groups balanced for hypersensitivity scores to tactile and airblast stimuli: (1) use of a dentifrice containing 0.3% triclosan, 2.0% PVM/NIA copolymer, small particle silica and 0.243% sodium fluoride (Test Dentifrice); (2) use of a conventional commercially-available dentifrice containing 0.3% triclosan, 2.0% PVM/MA copolymer and 0.243% sodium fluoride (Control Dentifrice).

Subjects were instructed to brush their teeth at home for one minute, twice daily (mornings and evenings), using only their assigned dentifrice product and provided soft-bristled toothbrush. Hypersensitivity examinations were repeated after four and eight weeks of product use.

Three hundred and fifty subjects completed all study visits. At both the four-week and the eight-week examinations, the Test Dentifrice group exhibited statistically significantly more favorable tactile hypersensitivity scores than did the Control Dentifrice group, with improvements of 11.5% and 17.9%, respectively. Additionally, at both the four-week and eight-week examinations, the Test Dentifrice group exhibited statistically significantly more favorable air blast hypersensitivity scores than did the Control Dentifrice group, with improvements of 16.1% and 23.3%, respectively.

The results of this clinical study support the conclusions that a dentifrice containing 0.3% triclosan, 2.0% copolymer, small particle silica and 0.243% sodium fluoride is efficacious in providing dentinal hypersensitivity reduction when used over a period of four and eight weeks as compared to the commercially available Control Dentifrice.

### Example 5

### Occlusion Efficacy and Enhanced Potassium Delivery of Compositions of the Invention

This experiment demonstrated the enhanced efficacy of oral compositions of the invention for dental sensitivity relief by use of a dentin tubule occlusion system in combination with conventional potassium desensitizing agents, and in particular, an oral composition of the invention comprising a silica having a particle size characterized by the parameters of a median particle size of about 2 μm to about 4 μm, a d₁₀ of about 0.5 μm to about 2 μm, and a d₉₀ of about 5 μm to about 10 μm, and a polymethylvinylether/maleic anhydride copolymer having a median molecular weight of 2,000,000 (“Test Dentifrice”) and the desensitizer potassium nitrate. For these experiments, “Composition E” (Table 4), was prepared and assayed.

TABLE 4
“Composition E”

| Ingredient | Wt % |
|---|---|
| Glycerin | 19.74 |
| Water | QS |
| Gantrez 13% solution | 15.00 |
| Sorbitol | 15.00 |
| Sorbosil AC43 silica | 10.00 |
| Thickening silica | 8.00 |
| Potassium nitrate | 5.00 |
| PEG600 | 3.00 |
| SLS | 1.50 |
| Sodium hydroxide 50% | 1.45 |
| Flavor | 1.10 |
| Sodium phosphate tribasic | 1.00 |
| Titanium dioxide | 1.00 |
| Poloxomer 407 | 1.00 |
| Saccharin | 0.40 |
| Sodium CMC | 0.40 |
| Sodium Fluoride | 0.243 |
| Xanthan Gum | 0.17 |

The most accepted explanation for dentinal hypersensitivity is based on Brännström's hydrodynamic theory, which postulates that various stimuli, such as cold, heat, pressure, acids, or sweets displace the fluid in the dentinal tubules inwardly or outwardly activating the nerve endings at the pulp/dentine interface, resulting in pain. In order to treat hypersensitivity, two major methodologies have been identified as efficacious: 1. Nerve desensitization by delivering potassium ions through dentin, and 2. Decrease of fluid movement by occlusion of dentinal tubules. Surprisingly, it was found that the combination of the small particle silica/polymer occlusion composition with a potassium desensitizing agent enhanced the delivery of potassium inward through the dentin tubules.

1. Confocal Microscopy

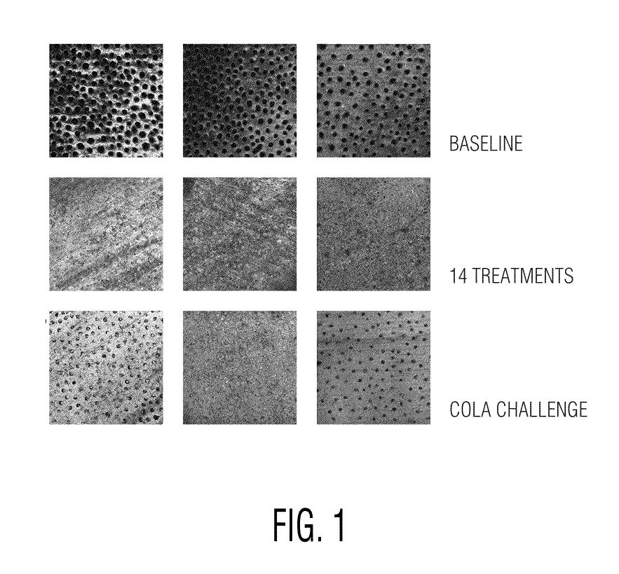

A dose-response study of the silica of the Test Dentifrice regarding occlusion efficacy was examined using confocal microscopy, at three different levels of silica particles, 5%, 7.5% and 10% (w/w). Ten percent silica particles showed superior occlusion effect after 14 brushing treatments and 2 acid-challenge exposures in comparison to conventional commercial desensitizing products. Dentin disks were treated 14 times to mimic a 7-day brushing regimen. Cola challenge was performed in order to have a clear distinction amongst the tested products. The confocal microscopy, images are displayed in FIG. 1 (zoom 50×50 μm), demonstrating superior protective/desensitizing effect of The Test Dentifrice. The occlusion effect of The Test Dentifrice is preserved after the cola challenge, as indicated by no changes on the surface image (see column 2 in FIG. 1).

2. Hydraulic Conductance

Hydraulic Conductance testing was performed to evaluate the degree of occlusion taking place on a dentin segment attached to a sensor that measures the displacement of water over time. The occlusion efficacy is related to a decrease in the hydraulic conductance, or water permeability of dentin segments after treatment with dentifrice. The baseline was measured on a dentin segment previously etched to have the maximum open tubules and higher permeability (“0% occlusion”). A decrease in the hydraulic conductance after treatment with dentifrice indicates the occlusion effect, calculated in the percentage of permeability reduction. The Test Dentifrice was shown to be 50% better than conventional commercial desensitizing products in this test.

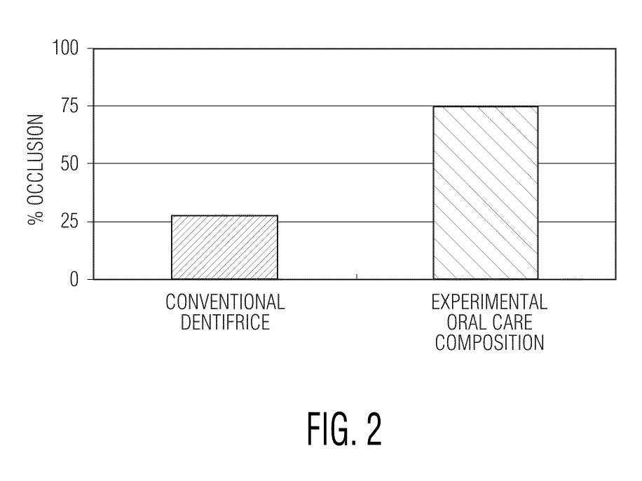

FIG. 2 illustrates the percentage of occlusion observed with each composition in comparison to its own baseline as determined using hydraulic conductance. This result is in agreement with the confocal microscopy results, namely, higher occlusion percentage is observed for the Test Dentifrice.

For Composition E, the hydraulic conductance testing procedure was modified to allow testing of an alternative application method for instant relief which involves rubbing/smearing the toothpaste onto a sensitive tooth with one finger. The surface of dentin segments was wetted with 10 μl PBS buffer to simulate a moist tooth in the mouth. Composition E was applied to the dentin segments undiluted (“neat”) with one finger and rubbed in a circular motion for 1 minute. The sample was rinsed with distilled, deionized water and the hydraulic conductance measured at 70 cm water pressure. The procedure was repeated using a conventional non-desensitizing dentifrice (Control Dentifrice, Table 5). Statistically significant (Student's t-test, p<0.05) lower conductance was observed for dentin treated with Composition E, indicating superior occlusion after only one treatment using a different method of application (i.e., other than conventional brushing) of a dentifrice of the invention. This superior occlusion results in a rapid desensitization of the tooth in comparison to conventional dentifrices and methods of application.

TABLE 5
Effect of Composition E on Dental Sensitivity.

| | % Flow vs. |
|---|---|
| | etched |
| Composition | baseline |
| Composition E | 31 ± 14 |
| Control | 88 ± 10 |
| Dentifrice | |

3. Potassium Flux

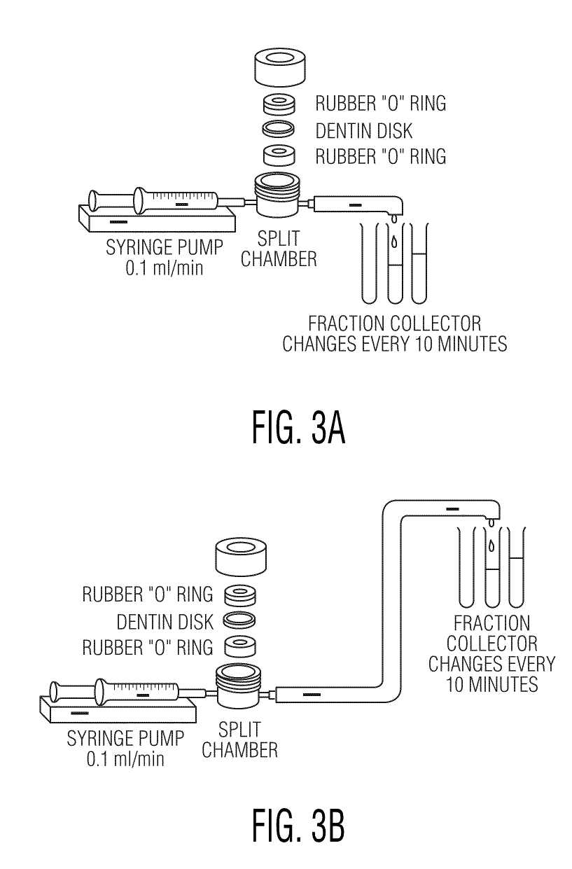

The potassium flux (“K flux”) methodology demonstrated the beneficial effect of occlusion agents on decreasing the outward fluid movement by blocking dentin tubules. The same pressure-blocking effect would help the inward flow of nerve a desensitization agent, such as potassium nitrate. A schematic illustration of the potassium flux experiment is depicted hi FIG. 3.

Human dentin disks were brushed With either composition E (Test Dentifrice or a conventional commercial desensitizing composition containing the same level of potassium nitrate) and mounted in a Pashley's split chamber that allows a constant flow of water to be collected at predetermined time intervals. Aliquots of potassium solution %% ere added to the top chamber on the treated dentin disk. The first set of fractions was collected at pressure zero (i.e., lower chamber is aligned with collector (3a), offering no resistance to the water flow, allowing the ions to diffuse through the dentin disks). Next, the collector was raised (3b) to simulate a pulpal pressure of 20 cm H₂0. The opposing pressure created by the height difference delayed the ion flour, resulting in lower concentration at the collector. Fractions were analyzed by HPLC for soluble potassium. Concentration values were converted to flux, as per Fick's law of diffusion, expressed here as ×10⁻¹⁰ mol/cm²·s. At the end of the experiment, the potassium flux was calculated to determine the relative efficacy of brushed disks on delivering potassium under pulpal pressure.

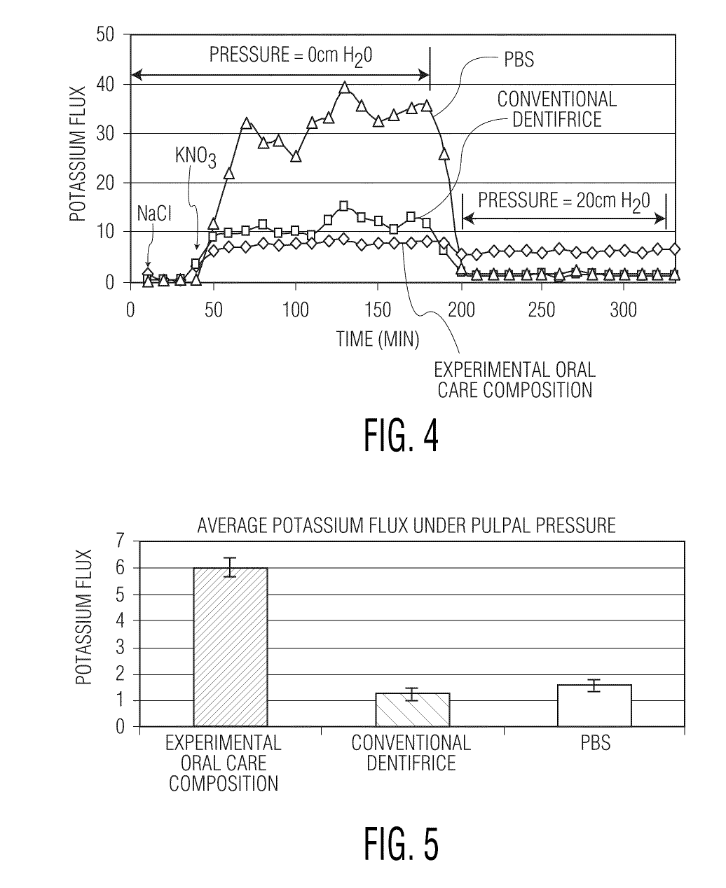

FIG. 4 shows a comparison of the two products investigated and a control. Each composition was evaluated in triplicate and one disk was studied per day. The experiment was carried out as follows: The dentin disk was mounted in the Pashley's split chamber and brushed respectively with either the Test Dentifrice, a conventional commercial dental desensitizing product, or Phosphate buffer (PBS). After brushing, the disk (in the chamber) was rinsed thoroughly and the chamber was connected to the experimental system. A constant water flow was provided by a syringe pump at 0.2 mL/minute and the fraction collector changed every 10 minute.

Initially, three fractions were collected after NaCl solution was added to the top chamber in order to rinse the system. In the fourth fraction, a respective potassium source was added, i.e., toothpaste slurry of the study products and KNO3 solution for PBS treated samples. Chamber and collector were aligned to have a final pressure of zero cm H₂0 for 18 fractions or 180 minutes. In this hypothetical condition, the occlusion efficacy can be inferred by the potassium diffusion (as ×10⁻¹⁰ mol/cm²·s), where the PBS-treated disk results in higher K flux due its lack of occlusion. After 18 fractions, the chamber is lowered with respect to the collector, simulating an in vivo situation, with a pulpal pressure of 20 cm H₂O. At this point, the potassium flow was expected to decrease due to the opposed pressure created by the difference of height chamber/collector. The disk which presents the smallest change or the higher potassium flux under the pulpal pressure will be more effective on delivering potassium ions to a tooth with exposed dentin tubules. An occlusion system that diminishes the negative effect of the water pressure on the ion diffusion will result in higher potassium flux at the end. The results in FIG. 4 show clearly that The Test Dentifrice presents the highest potassium flux under 20 cm H₂O in comparison to conventional commercial desensitizing product and PBS-brushed disks. A comparison of the average flux under pulpal pressure is plotted for the two studied products and PBS in FIG. 5.

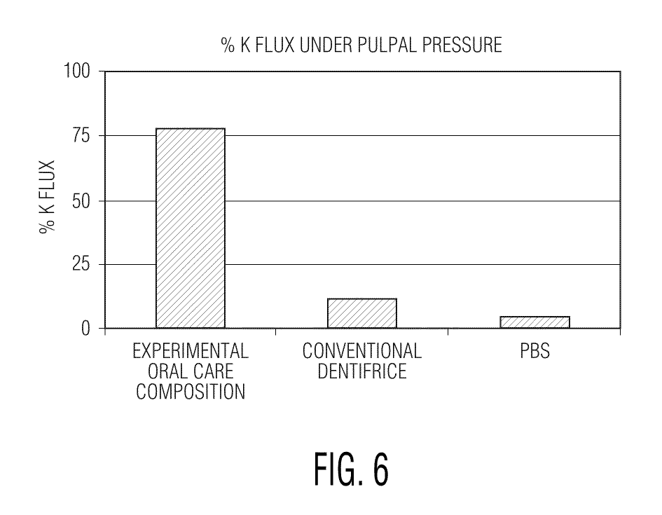

FIG. 6 illustrates the same trend in terms of percentage of potassium flux after the simulated pulpal pressure had been applied. These results suggest faster action of the Test Dentifrice in comparison to convention commercial desensitizing product.

The theory of having an efficient occlusion system to impede the detrimental action of the outward opposed fluid flow to the ion diffusion is confirmed is with this experiment, i.e. the occlusion provided by a composition of the present invention, the Test Dentifrice, is more effective on aiding potassium to diffuse across human dentin.

### Example 6

### AUC 3.95 Values Measured for Various Silicas

Table 6 illustrates the AUC 3.95 values measured for various silica-containing compositions and silica starting materials, including silica-containing compositions of the invention and silica starting materials used in silica-containing compositions of the invention.

TABLE 6
AUC 3.95 Values for Various Silicas and
Silica-Containing Compositions.

| SAMPLE | % AUC 3.95 SILICA | d₅₀ |
|---|---|---|
| Test Dentifrice (Examples 3 and 4) | 24.0 | 8.2 |
| Control Dentifrice (Example 3) | 16.4 | 10.6 |
| Control Dentifrice (Example 4) | 16.6 | 11.0 |
| INEOS AC43 Silica starting material | 49.4 | 3.95 |
| Conventional High-Cleaning Silica | 22.0 | 8.27 |
| Conventional Abrasive Silica | 14.4 | 11.46 |
| Conventional Thickening Silica | 2.8 | 14.97 |

1. An oral care composition comprising:
a. an adherent material; and
b. silica particles,
wherein the oral care composition provides a fluid flow rate of no greater than about 45% of the fluid flow rate of etched dentin.

2. A oral care composition comprising:
a. an adherent material; and
b. silica particles having a particle size distribution (PSD) of 2 μm to 5 μm,
wherein the oral care composition provides a fluid flow rate of no greater than about 45% of the fluid flow rate of etched dentin.

3. An oral care composition comprising:
a. an adherent material; and
b. silica particles having a median particle size of 2 μm to 5 μm,
wherein the oral care composition provides a fluid flow rate of no greater than about 45% of the fluid flow rate of etched dentin.

4. An oral care composition comprising:
a. an adherent material; and
b. silica particles having an average particle size of 2 μm to 5 μm,
wherein the oral care composition provides a fluid flow rate of no greater than about 45% of the fluid flow rate of etched dentin.

5. An oral care composition comprising:
a. an adherent material; and
b. silica particles having an average particle size of 2.7 μm to 4.0 μm,
wherein the oral care composition provides a fluid flow rate of no greater than about 45% of the fluid flow rate of etched dentin.

6. An oral care composition comprising:
a. an adherent material; and
b. a population of silica particles having a particle size selected from the group consisting of 2 μm, 2.5 μm, 3 μm, 3.5 μm, 4 μm, 4.5 μm, and 5 μm, wherein said population of silica particles comprise at least 20% of the total silica particles in said oral care composition,
wherein the oral care composition provides a fluid flour rate of no greater than about 45% of the fluid flow rate of etched dentin.

7. An oral care composition comprising:
a. an adherent material; and
b. silica particles having a median particle size of 3 μm to 5 μm, a d₁₀ of 1.5 μm to 3 μm, and a d₉₀ of 6 μm to 11 μm,
wherein the oral care composition provides a fluid flown, rate of no greater than about 45% of the fluid flow %, rate of etched dentin.

8. An oral care composition comprising:
a. an adherent material; and
b. silica particles having a median particle size of 2 μm to 4 μm, a d₁₀ of 0.5 μm to 2 μmil, and a d₉(o of 5 μm to 10 μm,
wherein the oral care composition provides a fluid flow rate of no greater than about 45% of the fluid flow rate of etched dentin.

9. An oral care composition comprising:
a. an adherent material; and
b. silica particles,
wherein the composition has a cumulative particle size volume (AUC 3.95) of at least 20%, and wherein the oral care composition provides a fluid flow rate of no greater than about 45% of the fluid flow rate of etched dentin.

10. An oral care composition comprising:
a. an adherent material; and
b. silica particles,
wherein the silica particles comprise a population of starting material silica particles having a cumulative particle size volume (AUC 3.95) of at least 40%, wherein the oral care composition provides a fluid flow rate of no greater than about 45% of the fluid flow rate of etched dentin.

11. The composition of claim 1, wherein the silica particles have a porosity of less than 0.45 cc/g in pores of 600 Angstroms or smaller.

12. The composition of claim 1, wherein adherent material is a polymer having a number average molecular weight between 100,000 and 2,500,000, inclusive.

13. The composition of claim 1, wherein the adherent material is selected from polymers of polyvinyl phosphonic acid, poly(l-phosphonopropene) sulfonic acid, poly(beta styrene phosphonic acid), alpha styrene phosphonic acid, synthetic anionic polymeric polycarboxylate, maleic anhydride, maleic acid, and methyl vinyl ether.

14. The composition of claim 1, wherein the adherent molecule is a polymer of methyl vinyl ether and maleic anhydride.

15. The composition of claim 1, wherein the composition is formulated into a form selected from a rinse, a paste, a gel, a gum, a dissolvable lozenge, and a film.

16. The composition of claim 1, wherein the composition is formulated into a form selected from a dissolvable film.

17. The composition of claim 1, further comprising a non-silica desensitizing agent.

18. The composition of claim 17, wherein the desensitizing agent is selected from the group consisting of a nitrate salt, an arginine ester, a bicarbonate salt, potassium nitrate, potassium chloride, an arginine-bicarbonate-phytate complex, potassium citrate, and arginine.

19. The composition of claim 1, further comprising an antibacterial agent.

20. The composition of claim 1, further comprising an agent selected from a chemical whitening agent, an opaque whitening agent and an anticalculus agent.

21. The composition of claim 1, further comprising 2,4,4′-trichloro-2′-hydroxydiphenyl ether.

22. The composition of claim 1, further comprising a surfactant system that comprises sodium lauryl sulfate and tauranol.

23. The composition of claim 1, further comprising a surfactant system that consists essentially of sodium lauryl sulfate and tauranol in a ratio of 1:5 to 1:3.

24. The composition of claim 1, further comprising an agent selected from a stannous ion agent; a fluoride compound; sodium fluoride; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; delmopinol; octapinol; nisin; zinc ion agent; copper ion agent; essential oils; furanones; bacteriocins, ethyllauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, and propolis.

25. A method of reducing dental sensitivity comprising applying to the surface of a mammalian tooth an oral care composition of claim 1.

26. A method of reducing dental sensitivity comprising applying to the surface of a mammalian tooth an oral care composition of claim 1, wherein the adherent material is selected from polymers of polyvinyl phosphonic acid, poly(l-phosphonopropene)sulfonic acid, poly(beta styrene phosphonic acid), alpha styrene phosphonic acid, synthetic anionic polymeric polycarboxylate, maleic anhydride, maleic acid, and methyl vinyl ether.

27. A method of reducing dental sensitivity comprising applying to the surface of a mammalian tooth an oral care composition of claim 1, wherein the particles have a porosity of less than 0.45 cc/g in pores of 600 Angstroms or smaller.

28. A method of protecting dental from acid-mediated degradation, comprising applying to the surface of a mammalian tooth an oral care composition of claim 1.

29. A method of maintaining or increasing the systemic health of a mammal comprising applying a composition to an oral surface of a man mal at least once a day for a duration of time, wherein the composition comprises:
a. an oral care composition of claim 1, wherein the silica particles are present in the composition in an amount of 5% by weight or greater, and
b. an agent selected from triclosan; triclosan monophosphate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4ethylpyridinium chloride (TDEPC); octenidine; delmopinol; octapinol; nisin; zinc ion agent; copper ion agent; essential oils; furanones; bacteriocins, ethyllauroyl arginate, extracts of magnolia, a metal ion source, fluoride, stannous ions, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creative, and propolis.

30. A method of occluding a dentin tubule within the surface of a mammalian tooth comprising applying to the tooth surface a composition comprising an adherent material and a silica particle having a median particle size of no greater than a dentin tubule.

31. A method of occluding a dentin tubule within the surface of a mammalian tooth comprising applying to the tooth surface the composition of claim 1.

32. The method of claim 31, wherein the method of application is a method other than brushing the tooth surface.

33. A method of desensitizing a tooth in less than one day, the method comprising applying to the tooth surface the composition of claim 1.

34. A method of increasing the potassium flux through one or more dentin tubules, the method comprising applying to a tooth surface the composition of claim 1.

35. A method of increasing the potassium flux of a conventional potassium-containing desensitizing dentifrice, the method comprising applying to the tooth surface the composition of claim 1, wherein the composition is applied:
(a) prior to application of the conventional dentifrice to the tooth,
(b) concomitant with application of the conventional dentifrice to the tooth, or
(c) concomitant with application of the conventional dentifrice to the tooth in a mixture with the conventional dentifrice,
or by way of any combination of (a)-(c).
